(19) United States
(12) Patent Application Publication
(10) Pub. No.: US 2005/0256466 A1
(43) Pub. Date: Nov. 17, 2005

(54) Methods and apparatus for concealing ostomy pouch

(76) Inventor: Rocky Winkler, Glendale, AZ (US)

Correspondence Address: Noblitt & Gilmore, LLC, Suite 6000, 4800 North Scottsdale Road, Scottsdale, AZ, 85251, US

(21) Appl. No.: 10/837,533

(22) Filed: Apr. 30, 2004

(51) Int. Cl. A61F 5/44

(52) U.S. Cl. 604/337; 604/332; 604/342

(57) ABSTRACT

Methods and apparatus for concealing an ostomy pouch according to various aspects of the present invention include a support and a pouch cover. The pouch cover provides a cover over the ostomy pouch. The support is configured to be attached to the pouch cover and/or the ostomy pouch to retain the position of the pouch cover and/or ostomy pouch.

### FIELD OF THE INVENTION

The invention relates to methods and apparatus for covering and/or holding ostomy pouches.

### BACKGROUND OF THE INVENTION

Many people lose the normal function of the bladder or bowel due to birth defects, disease, injury or other disorders. To address the problem, doctors may perform an ostomy, such as a colostomy, ileostomy, or urostomy. Such procedures facilitate expulsion of bodily wastes through a surgically-created opening, typically formed through the abdominal wall. In a colostomy, a portion of the colon is moved to the body exterior. This new opening in the bowel, the stoma, allows stool to pass directly out of the body, bypassing a diseased or damaged section of the colon.

Ostomates lose voluntary control of bowel movements. Some ostomies expel waste without intervention, while others may be controlled by the ostomate. To capture waste, most ostomates wear ostomy pouches or bags attached over the stoma at all times. Ostomies are often psychologically difficult to deal with, and the visible pouch may cause embarrassment or shame. Consequently, ostomates may be inclined to avoid situations that could lead to exposing the colostomy pouch to avoid embarrassment.

### SUMMARY OF THE INVENTION

Methods and apparatus for concealing an ostomy pouch according to various aspects of the present invention include a support and a pouch cover. The pouch cover provides a cover over the ostomy pouch. The support is configured to be attached to the pouch cover and/or the ostomy pouch to retain the position of the pouch cover and/or ostomy pouch.

### BRIEF DESCRIPTION OF THE DRAWINGS

A more complete understanding of the present invention may be derived by referring to the detailed description when considered in connection with the following illustrative figures. In the following figures, like reference numbers refer to similar elements and steps.

FIG. 1 is an illustration of an ostomy pouch concealment system according to various aspects of the present invention;

FIGS. 2A-B are illustrations of the front and rear sides of an exemplary support;

FIG. 3 is an illustration of an exemplary pouch cover; and

FIGS. 4A-B are illustrations of a wearer wearing the ostomy pouch concealment system.

Elements and steps in the figures are illustrated for simplicity and clarity and have not necessarily been rendered according to any particular sequence. For example, steps that may be performed concurrently or in different order are illustrated in the figures to help to improve understanding of embodiments of the present invention.

### DETAILED DESCRIPTION OF EXEMPLARY EMBODIMENTS

The present invention is described partly in terms of functional components and various assembly and/or operating steps. Such functional components may be realized by any number of components configured to perform the specified functions and achieve the various results. For example, the present invention may employ various elements, materials, configurations, pouches, supports, pouch covers, straps, and the like, which may carry out a variety of functions. In addition, the present invention may be practiced in conjunction with any number of applications, environments, and ostomy pouches, and the systems and components described are merely exemplary applications for the invention. Further, the present invention may employ any number of conventional techniques for manufacturing, assembling, wearing, and the like.

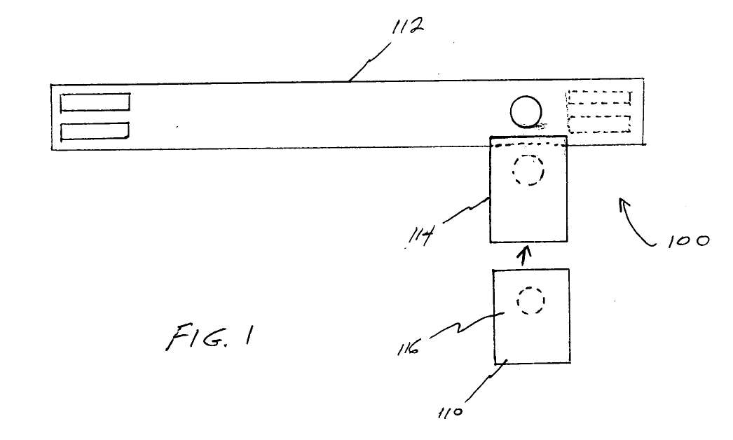

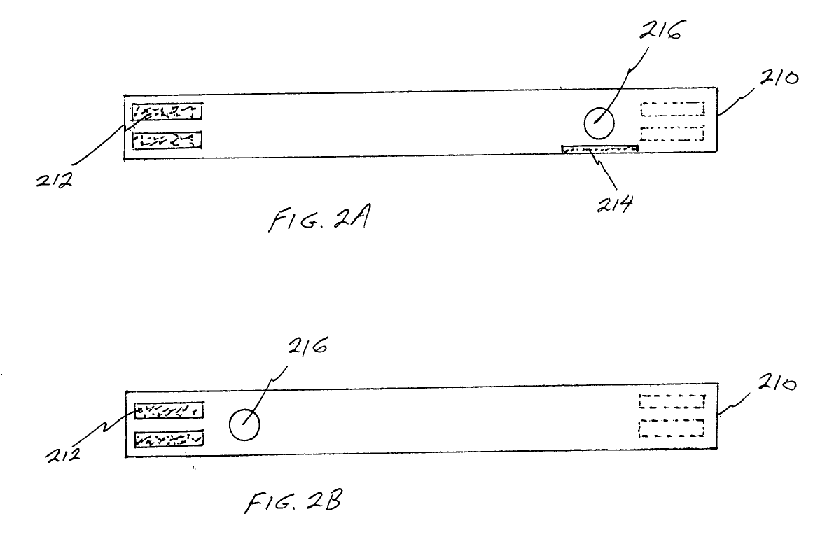

Referring now to FIG. 1, an ostomy pouch concealment system 100 for containing and/or camouflaging an ostomy pouch 110 according to various aspects of the present invention may be implemented in conjunction with a support 112 and a pouch cover 114. The pouch cover 114 covers and/or contains a pouch, such as the ostomy pouch 110. The support 112 is connected to the pouch cover 114 for supporting the pouch cover 114. The support 112 is configured to be attached to another object, such as a person.

More particularly, the support 112 suitably comprises a support system for holding the pouch cover 114 approximately in a desired position. The support 112 may comprise any suitable system, such as a strap, belt, fastener, band, or brace, and may be configured to attach to any appropriate position, such as the shoulder, waist, leg, pelvis, chest, or back, for example according to the position of the person's particular stoma. In the present embodiment, the support 112 is configured for a colostomy pouch 116. The colostomy pouch 116 fits over the wearer's stoma, which may be located in any appropriate position, for example on the lower abdomen near the hip. The support 112 may comprise any suitable mechanism for supporting the pouch cover 114 in the area of the colostomy pouch 116, such as an adjustable strap to be worn around the waist.

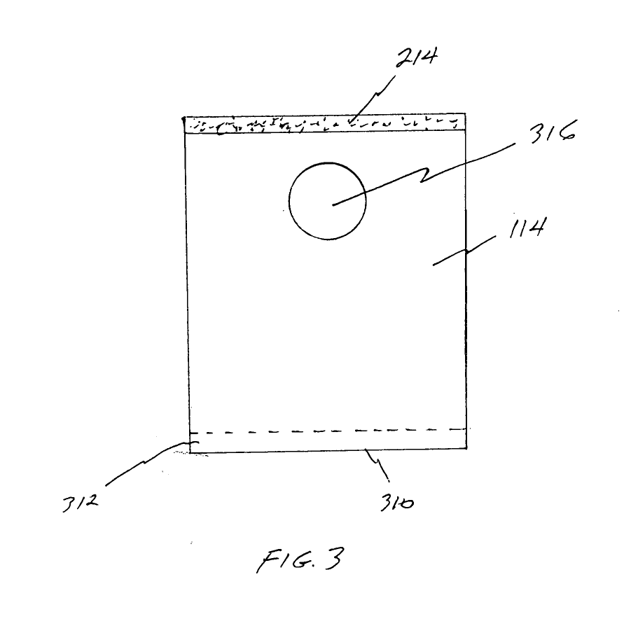

For example, referring to FIGS. 2A-B, the support 112 for the present colostomy pouch concealment system 100 comprises a belt 210 to be worn by the wearer and connected to the colostomy pouch 116 and/or colostomy pouch cover 114. The belt 210 may comprise any suitable material for wear by the wearer and supporting the colostomy pouch 116 or pouch cover 114. For example, the belt 210 may comprise a strip of fabric or other flexible material. In the present embodiment, the belt 210 is configured to be worn against the skin and under the clothes of the wearer. The present belt 210 is suitably made of a biosensitive material to avoid irritating the skin of the wearer. In addition, the belt 210 is suitably made of a lightweight and relatively durable material, such as cotton, silk, hide, synthetic, blended natural and/or synthetic fibers, or other suitable fabric or material. The belt 210 may also be washable for easy cleaning, and may also be configured or treated to be stain-resistant, water-resistant, and/or sweat-resistant. In the present embodiment, the belt 210 comprises a treated textile fabric, such as a stain-resistant and water-repellant textile fabric, such as a polyester fabric treated with one or more fluorochemical treating agents, polymeric and/or copolymeric compositions, interpenetrating polymers, antimicrobial agents, and/or crosslinkable resins on at least one side of the fabric. For example, the belt may comprise a Crypton Super Fabric available from Hi-Tex, Inc., or other suitable fabric.

The belt 210 may be configured in any suitable manner to be worn by the wearer. For example, the strip of material may have any appropriate dimensions desired by the wearer. An exemplary belt 210 for a wearer having a 38-inch waist may comprise a strip of material that is about 40 inches long and about four inches wide. The dimensions of the belt 210 may be configured according to the size and preferences of the wearer, the area in which the belt 210 is to be worn, and the like. In addition, the belt 210 may comprise an elastic material to provide a snug fit and accommodate variations in size. Further, the belt 210 may include any desired patterns or colors, for example to allow the wearer to match different colors to different clothes. For example, the belt 210 may have different colors on the two main surfaces so that the belt 210 is reversible to match different clothes.

The belt 210 suitably includes a fastener 212 for fastening the belt 210 around the wearer's body. The belt 210 is also suitably adjustable for the comfort of the wearer. In the present embodiment, the adjustability function is incorporated into the fastener 212, such as by using a hook-and-loop fastener. The fastener 212 may comprise any suitable fastener, however, such as one or more hooks, buttons, zippers, buckles, or snaps. Similarly, the adjustability function may be implemented in any suitable manner, such as using elastic material or other adjustable closures.

The belt 210 is also suitably configured to connect to at least one of the pouch and the pouch cover 114. The belt 210 may be attached to the pouch and/or pouch cover 114 using any suitable mechanism. For example, in the present embodiment, the belt 210 includes a pouch cover fastener 214, such as one element of a hook-and-loop fastener or a button, and the other element of the hook-and-loop fastener or button hole is attached to or formed in the pouch cover 114. The pouch cover fastener 214 may comprise, however, any suitable fastener for attaching the pouch cover 114 (and/or the ostomy pouch 110) to the belt 210.

The belt 210 may be further configured for the particular application or needs of the wearer. For example, the belt 210 is suitably configured to accommodate a stoma on either side of the body. In the present embodiment, the belt 210 may be rotated around the body to accommodate the position of the stoma. In addition, the belt 210 may be inverted or turned inside-out to optimize the location of the belt fastener 212 and the pouch cover fastener 214 with respect to the stoma according to the wearer's convenience.

In addition, the belt 210 may be worn around the wearer's body at the same height as the stoma, instead of above the stoma. To facilitate access to the ostomy pouch 110, the belt 210 may include a hole 216 so that the stoma may pass through the belt 210 to the ostomy pouch 110 and pouch cover 114. In this configuration, the pouch cover fastener 214 is suitably worn at the top of the belt 210 to retain the pouch cover 114 in position. For a lower stoma, the belt 210 may be worn with the fastener 214 on the bottom of the belt 210 and above the pouch cover 114 to maintain the position of the pouch cover 114.

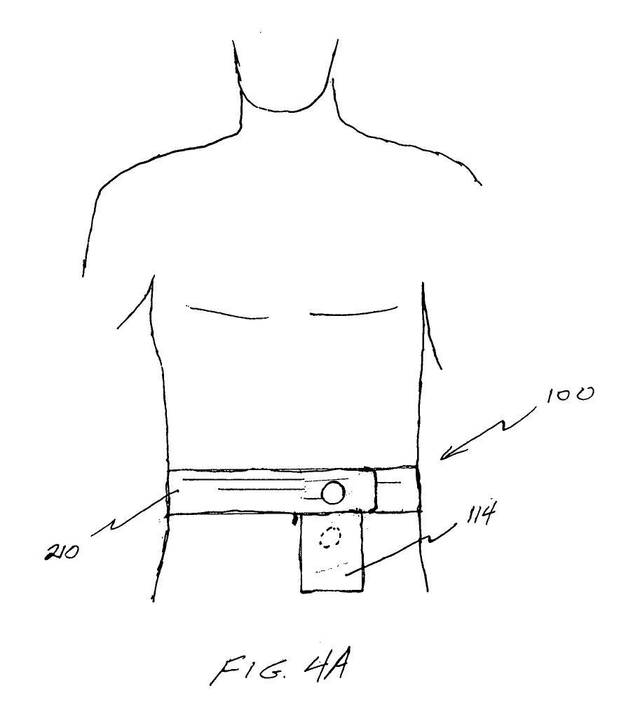

Referring now to FIG. 3, the pouch cover 114 is suitably configured to cover the ostomy pouch 110. The pouch cover 114 may be configured in any suitable manner to cover the ostomy pouch 110, for example by forming an envelope, shroud, sack, wrap, or other full or partial cover around the ostomy pouch 110. The pouch cover 114 may be retained in position relative to the ostomy pouch 110 in any suitable manner, such as by connecting the pouch cover 114 to the ostomy pouch 110 and/or the support 112.

In the present embodiment, the pouch cover 114 is configured to cover the colostomy pouch 116 and may be connected to the belt 210. The pouch cover 114 may comprise any suitable material and configuration for attachment to the belt 210 and at least partially covering the colostomy pouch 116. For example, the pouch cover 114 may comprise an envelope or other enclosure of fabric or other flexible material, and may comprise the same material as the belt 210. In addition, the pouch cover 114 may be colored to match or complement the belt 210. The pouch cover 114 is suitably made of a biosensitive material, and, like the belt 210, may comprise a lightweight and relatively durable material, such as cotton, silk, hide, synthetic, blended natural and/or synthetic fibers, or other suitable fabric or material. Likewise, the pouch cover 114 is suitably washable for easy cleaning, and may also be configured or treated to be stain-resistant, water-resistant, and/or sweat-resistant. In the present embodiment, the pouch cover 114 comprises a stain- and water-resistant fabric, such as a Crypton Super Fabric.

In the present embodiment, the pouch cover 114 is configured to substantially envelop the ostomy pouch 110. The pouch cover 114 may be configured in any suitable manner to cover the ostomy pouch 110, such as by forming a partial or full cover over the ostomy pouch 110. For example, the present pouch cover 114 may comprise an enclosure configured to fit around the colostomy pouch 116. The pouch cover 114 comprises a cloth pouch shaped to accommodate the colostomy pouch 116, such as a particular standard size of colostomy pouch 116. Different pouch covers 114 may be utilized for different sizes and shapes of colostomy pouches.

The present colostomy pouch cover 114 is configured to receive the colostomy pouch 116. The mechanism for inserting the colostomy pouch 116 into the pouch cover 114 may comprise any appropriate mechanism, such as a hole formed in the pouch cover 114 or a flap that may be wrapped over the colostomy pouch 116. The present colostomy pouch 116 includes an opening 310 formed in the pouch cover 114, for example at one end or both ends of the pouch cover 114, that is wide enough to receive a particular type or size of colostomy pouch 116. The opening 310 is suitably formed along the entire length of the end of the pouch cover 114 to facilitate insertion and removal of the colostomy pouch 116 from the pouch cover 114.

The opening 310 may also be configured to be closed or otherwise configured to secure the colostomy pouch 116 within the pouch cover 114. Any suitable mechanism may be employed to secure the colostomy pouch 116 within the pouch cover 114, such as a zipper, button, snap, or other suitable closure. In the present embodiment, the pouch cover 114 includes a hook-and-loop fastener 312, for example lining the opening. Thus, after the colostomy pouch 116 is inserted into the pouch cover 114, the opening 310 may be closed to fully cover the colostomy pouch 116 and retain the colostomy pouch 116 within the pouch cover 114.

The pouch cover 114 may also include an attachment mechanism for attaching the pouch cover 114 to the support 112. The pouch cover attachment mechanism may comprise any appropriate mechanism for connecting the pouch cover 114 to the support 112, including a permanent or removable connection. In the present embodiment, the pouch cover attachment mechanism comprises the hook-and-loop fastener 214 configured to removably attach the pouch cover 114 to the belt 210. For example, referring now to FIGS. 2A-B and 3, one portion of the hook-and-loop fastener 214 may be connected to an edge of the belt 210 and the other portion connected to an edge of the pouch cover 114, such as the edge of the pouch cover 114 opposite the opening.

The pouch cover 114 is also suitably configured to accommodate connection of the ostomy pouch 110 to the wearer's body around the stoma. For example, the pouch cover 114 may be configured to at least partially expose the ostomy pouch 110 where the ostomy pouch 110 is configured to connect around the stoma. To facilitate connection of the colostomy pouch 116 to the wearer's body, the pouch cover 114 of the present embodiment includes a hole 316 formed in the side of the pouch cover 114 positioned to substantially coincide with the stoma connection point in the colostomy pouch 116. Thus, the colostomy pouch 116 may be connected to the wearer's body around the stoma through the hole in the pouch cover 114. The hole 316 in the pouch cover 114 may be configured in any size, shape, and/or position to accommodate connection of the colostomy pouch 116 around the stoma.

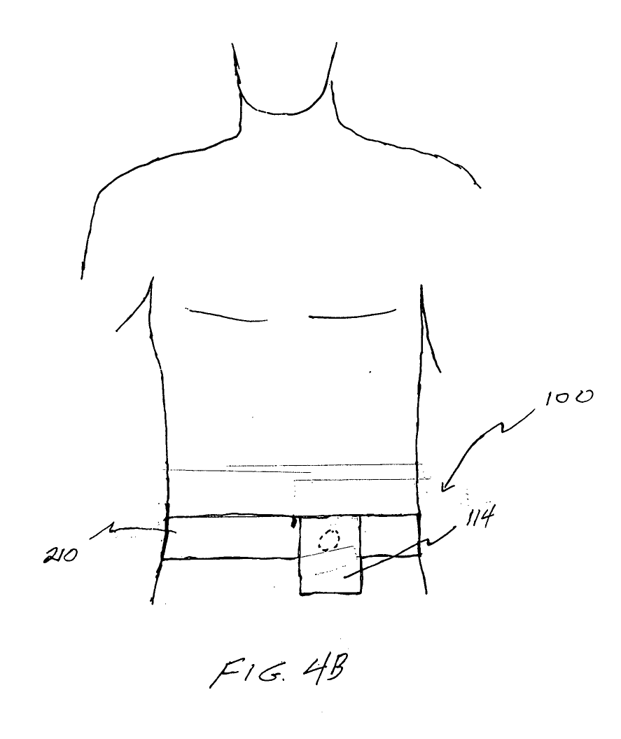

Referring to FIGS. 4A-B, to wear and use the ostomy pouch concealment system 100, the wearer straps the support 112 to his body near the location of the stoma. If the support 112 includes a hole to facilitate access to the stoma, the wearer may position the hole in the support 112 over the stoma (FIG. 4B). In the present system, the wearer may wrap the belt 210 around the wearer's waist and adjust the fit using the hook-and-loop fastener. If the wearer prefers to wear the belt 210 lower, the belt 210 may be worn lower with the hole in the belt 210 aligned with the stoma (FIG. 4B).

The ostomy pouch 110 may be fitted within the pouch cover 114. In the present embodiment, the colostomy pouch 116 may be inserted into the pouch cover 114 by sliding the colostomy pouch 116 through the opening 310 along the side of the pouch cover 114. The connection point for the colostomy pouch 116 is suitably aligned with the hole 316 in the pouch cover 114 to allow the colostomy pouch 116 to be connected to the wearer's stoma through the pouch cover hole 316. The colostomy pouch 116 may then be secured in position by closing the closing mechanism, such as the hook-and-loop fastener 312. The colostomy pouch 116, disposed within the pouch cover 114, may then be connected to the wearer's stoma. The wearer's remaining clothes may be worn over the ostomy pouch concealment system 100.

While the ostomy pouch concealment system 100 is being worn, it may be normally concealed from view under the wearer's clothes. For example, the present belt 210 and colostomy pouch cover 114 are suitably worn beneath the wearer's shirt and possibly pants or skirt. Occasionally, however, the ostomy pouch concealment system 100 may be visible, such as when the wearer moves in such a way that the clothes expose the ostomy pouch concealment system 100. Alternatively, the ostomy pouch concealment system 100 may be worn uncovered. In either case, the pouch cover 114 at least partially conceals the ostomy pouch 110 while it is exposed, improving the aesthetic appearance of the ostomy pouch 110. If the wearer wishes to empty the ostomy pouch 110 or release gas, the wearer has easy access to the ostomy pouch 110 via the opening 310. In addition, the wearer may exchange ostomy pouches 110 by removing the ostomy pouch 110 and pouch cover 114 from the support 112, replacing the original ostomy pouch 110 within the pouch cover 114 with a fresh one, attaching the fresh ostomy pouch 110 around the stoma, and connecting the pouch cover 114 to the support 112.

The particular implementations shown and described are illustrative of the invention and its best mode and are not intended to otherwise limit the scope of the present invention in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

The present invention has been described above with reference to a preferred embodiment. However, changes and modifications may be made to the preferred embodiment without departing from the scope of the present invention. These and other changes or modifications are intended to be included within the scope of the present invention.

1. A system for concealing an ostomy pouch borne by a wearer, comprising:
a support, wherein the support includes a fabric that is stain-resistant and water-resistant and is configured to be worn by the wearer; and
a pouch cover configured to be connected to the support and at least partially cover the ostomy pouch.

2. A system for concealing an ostomy pouch according to claim 1, wherein the support comprises a belt configured to be strapped to the wearer.

3. A system for concealing an ostomy pouch according to claim 2, wherein the belt is adjustable.

4. (canceled)

5. A system for concealing an ostomy pouch according to claim 1, wherein the pouch cover includes a hole formed in a surface of the pouch cover to facilitate access between the wearer and the ostomy pouch.

6. A system for concealing an ostomy pouch according to claim 1, wherein the support includes a hole formed therethrough to facilitate access between the wearer and the ostomy pouch.

7. A system for concealing an ostomy pouch according to claim 1, wherein the pouch cover is configured to be removably connected to the support.

8. A system for concealing an ostomy pouch according to claim 7, wherein the pouch cover includes a hook-and-loop fastener configured to fasten the pouch cover to the support.

9. A system for concealing an ostomy pouch according to claim 1, wherein the pouch cover is configured to removably receive the ostomy pouch via an opening formed in the pouch cover.

10. A colostomy pouch concealment system for concealing a colostomy pouch to be worn by a wearer, comprising:
a belt configured to be secured around a torso of the wearer;
a pouch cover configured to at least partially cover the colostomy pouch, wherein the pouch cover includes:
a fabric that is stain-resistant and water-resistant;
a surface having a hole defined therethrough configured to provide access between the wearer and the interior of the colostomy pouch; and
an opening configured to removably receive the colostomy pouch; and
a fastening mechanism for removably fastening the pouch cover to the belt.

11. A colostomy pouch concealment system according to claim 10, wherein the belt comprises:
a fabric strip having a first end and a second end; and
a hook-and-loop fastener configured having a first portion attached to the first end of the fabric strip and a second end attached to the second end of the fabric strip.

12. A colostomy pouch concealment system according to claim 10, wherein the belt is adjustable.

13. (canceled)

14. A colostomy pouch concealment system according to claim 10, wherein the belt includes a hole formed therethrough to facilitate access between the wearer and the colostomy pouch.

15. A colostomy pouch concealment system according to claim 10, wherein the fastening mechanism includes a hook-and-loop fastener.

16. A method of concealing an ostomy pouch to be worn by a wearer, comprising:
inserting the ostomy pouch into a pouch cover;
securing a support to the wearer, wherein the support comprises a fabric that is stain-resistant and water-resistant;
attaching the ostomy pouch to the wearer; and
attaching at least one of the ostomy pouch and the pouch cover to the support.

17. A method of concealing an ostomy pouch according to claim 16, wherein the support comprises a belt configured to be strapped to the wearer.

18. A method of concealing an ostomy pouch according to claim 17, wherein the belt is adjustable.

19. (canceled)

20. A method of concealing an ostomy pouch according to claim 16, wherein the pouch cover includes a hole formed in a surface of the pouch cover to facilitate access between the wearer and the ostomy pouch.

21. A method of concealing an ostomy pouch according to claim 16, wherein the support includes a hole formed therethrough to facilitate access between the wearer and the ostomy pouch.

22. A method of concealing an ostomy pouch according to claim 16, wherein the at least one of the ostomy pouch and the pouch cover is configured to be removably connected to the support.

23. A method of concealing an ostomy pouch according to claim 22, wherein the at least one of the ostomy pouch and the pouch cover includes a hook-and-loop fastener configured to fasten the at least one of the ostomy pouch and the pouch cover to the support.

24. A method of concealing an ostomy pouch according to claim 16, wherein the pouch cover is configured to removably receive the ostomy pouch via an opening formed in the pouch cover.
